(19) United States
(12) Patent Application Publication
(10) Pub. No.: US 2020/0253692 A1
(43) Pub. Date: Aug. 13, 2020

(54) SYSTEM AND METHOD FOR PRODUCTION OF DENTAL RESTORATIONS

(71) Applicant: Advanced Crown Technology, Inc., Harrisburg, PA (US)

(72) Inventor: Victor J. Hajjar, Harrisburg, PA (US)

(21) Appl. No.: 16/865,331

(22) Filed: May 2, 2020

Related U.S. Application Data
(63) Continuation-in-part of application No. PCT/US18/59145, filed on Nov. 5, 2018.
(60) Provisional application No. 62/581,109, filed on Nov. 3, 2017.

(51) Int. Cl. A61C 5/77; A61C 9/00; A61C 5/30; A61C 13/00; G16H 10/60; G16H 40/20; G05B 19/4155

(52) U.S. Cl. CPC A61C 5/77; A61C 9/0053; A61C 5/30; A61C 13/0004; G06Q 50/04; G16H 10/60; G16H 40/20; G05B 19/4155; A61C 13/0022

(57) ABSTRACT

The present invention is drawn to methods and means for easily, quickly and inexpensively producing one or more dental restorations—suited to each individual patient's needs, and having high quality and excellent fit—thereby reducing fit-issues and consequent rejections by patients. This is accomplished by a process which includes, scanning tooth/teeth of interest to create an “external prep” data file, preparing the tooth/teeth of interest for the dental restoration, scanning the prepared tooth/teeth of interest and saving this in an “internal prep” data file, volumetrically registering the “external scan” and the “internal scan” data files to each other in “digital space,” shaping both the inside and outside a generic dental restoration blank shell based on the “external prep” and “internal prep” data files to produce a patient-specific dental restoration, and then fastening the prepared dental restoration to the tooth/teeth of interest. This invention thereby provides the best possible outcomes, and an overall better patient experience.

### CROSS-REFERENCE TO RELATED APPLICATIONS

This application is a continuation-in-part of PCT Application No. PCT/US18/59145, filed on Nov. 5, 2018, which claims priority to U.S. Provisional Patent Application No. 62/581,109, filed on Nov. 3, 2017, both of which are incorporated herein by reference in their entireties.

### FIELD OF THE INVENTION

The present invention relates to new methods and means for production of improved dental restorations.

### BACKGROUND OF THE INVENTION

There has long been a need for dental restorations that are: easily and inexpensively manufactured, suited to each patient's individual needs, which provide high quality and excellent fit. Examples of past attempts to achieve these advantages include the following:

It is known from U.S. Pat. No. 5,549,476 issued Aug. 27, 1996 to Stern that dental restorations (e.g. crowns) may be made by a process which includes: taking a first impression of a tooth area to be restored including a prepared tooth in order to form a negative of the desired final “tooth shape”, placing a second impression material in the first impression, in the area of the prepared tooth, the first impression now containing the second impression material is placed over the prepared tooth, producing a “second impression mold” (i.e. a model), which has the shape of the final restoration, but is not a durable material, trimming the element intraorally, the second impression is replaced over the tooth and the occlusion is adjusted in the mouth, scanning the second impression mold, computer controlled milling is performed, and the finished restoration is cemented.

It is also known from U.S. Pat. No. 8,973,269 issued to Johnson et al. (hereinafter “Johnson '269”) to make dental appliances, which broadly may encompass: providing a first digital surface representation (e.g. digital data capture such as scanning), forming a first article of a first material having a desired outer shape (e.g. fabricating the desired outer shape of a dental appliance, removing an inner portion from said article to form a cavity (e.g. making an internal cutback), and filling said cavity in said article, to create a dental appliance such as a crown. More specifically, Johnson '269 may employ a model (e.g. of the patient's mouth) or a dental impression (e.g. of the patient's mouth), and scanning said model or impression, and further teaches that fabrication may take place at a dental laboratory or at a dentist's office or any other facility.

It is further known from U.S. Pat. No. 8,844,139 issued Sep. 30, 2014 to Johnson et al. to employ a process basically analogous to Johnson '269, but which encompasses workflows used to create layered, anatomical, all-ceramic dental appliances (such as a crown), which includes employing a model (e.g, of the patient's mouth) or a dental impression (e.g. of the patient's mouth) and scanning said model or impression, and that fabrication may take place at a dental laboratory or at a dentist's office or any other facility.

U.S. Pat. No. 7,333,874 issued Feb. 19, 2008 to Taub et al, is drawn to a method and system for designing and producing dental prostheses and appliances—which focuses on a “dental service center” which is part of a system comprising: at least one dental clinic, at least one dental laboratory, and the “dental service center” which is a separate entity from the dental laboratory (either the dental service center or dental lab being equipped to generate a virtual 3D model of the patient's teeth). Further this patent discloses utilizing a dental laboratory outside the dental clinic, scanning a patient's teeth, and transmitting the scanning data from the dental clinic to the lab and/or service center.

U.S. Pat. No. 8,483,857 to Orth (issued Jul. 9, 2013) discloses a method for producing dental prosthetics providing the advantage of only requiring a small assortment of blanks, using: a tooth database comprising at least one 3D model of a dental prosthetic, and selecting from said database a 3D model (design) which satisfies the demands/requirements and approximates the designed sub-region of the planned prosthetic item.

The “Apparatus and Method for Producing a Dental Prosthetic” family, which includes: WO 2001060277, CA2400383A1, EP 1255503A2, EP1255503A4, and U.S. Pat. No. 6,527,550, is drawn to a method for producing a dental prosthetic comprising the steps of: providing a dental prosthetic model and a dental prosthetic blank having exterior dimensions matched to those of said dental prosthetic model, placing said dental prosthetic model and said dental prosthetic blank in holding fixtures for machining said dental prosthetic blank; moving said holding fixture of said dental prosthetic blank in a single rotary axis; and moving a cutting tool supported on a linear rotary axis to mill said dental prosthetic blank—and a device for carrying out this method.

The “Method and apparatus for tooth restoration” family, which includes: U.S. Pat. Nos. 5,813,859, 6,190,171, 6,641,340, and WO1998032392A1 are drawn to a process for producing a dental prosthetic comprising the steps of providing a prosthetic model (e.g. selecting said prosthetic model from among a group of such models e.g. to select size and shape), providing a prosthetic blank having exterior dimensions matched to those of said prosthetic model (e.g. selecting said prosthetic blank from among a group of such blanks e.g. to select size and shape), forming an interior of said prosthetic model (e.g. by placing formable material therein, and placing the model onto a prepared tooth, and then curing said material) as a template; and matching an interior of said prosthetic blank to said prosthetic model.

U.S. Pat. No. 5,575,656 “Method and Apparatus for Tooth Restoration” which is drawn to a process (and associated device) for restoring a tooth, comprising the steps of removing a first portion of said tooth to reduce exterior dimensions of the tooth, and removing a second portion of said tooth to form an interior of said tooth into a predetermined shape for receiving artificial tooth material, wherein said step of removing said second portion further includes steps of placing a tooth template having a drill guide mounted thereon over said tooth, and lowering a milling blade through said drill guide to remove said second portion—said milling blade having predetermined dimensions for forming said predetermined shape.

All documents cited herein are hereby incorporated by reference.

### SUMMARY OF THE INVENTION

Objects of the subject invention include production of dental restorations that are: easily, quickly and inexpensively manufactured, suited to each individual patient's needs, which provide high quality and excellent fit thereby reducing fit-issues and consequent rejections by patients—and thereby providing the best possible outcomes and an overall better patient experience.

In accordance with one embodiment of the present invention, one or more dental restorations are produced by a method which includes: fabricating in a manufacturing facility generic dental restorations which are a shell with greater height than is anticipated to be needed and which may include/define a specialized tab/dog-bone and two sets of crossing/intersecting interior grooves and ridges (i.e. cross-hatching, or rugae), sending these generic dental restorations from the manufacturer to a dental practitioner's office/facility, scanning the tooth or teeth of interest and its vicinity using “means for scanning,” creating an “external prep” or “pre-scanning” data file which includes a “digital repair” of the tooth or teeth of interest, preparing/prepping the tooth/teeth of interest by forming into a desired shape for the dental restoration to attach to, scanning the prepped tooth/teeth of interest and saving this as an “internal prep” data file, volumetrically registering the “external scan” and the “internal scan” to each other in digital space, mounting the generic dental restoration into a suitable means for shaping, and loading the “external prep” and “internal prep” files into the means for shaping, using the “digital repair” design to select a suitable generic dental restoration and then control the means for shaping to shape both the exterior and interior of the dental restoration, removing the prepared modified restoration from the means for shaping, removing the specialized tab from the dental restoration, polishing the dental restoration, and fastening together the prepared, patient specific dental restoration and the prepared tooth/teeth of interest.

Another embodiment of the present invention is a kit which may be used to achieve the aforementioned objects, which kit comprises: a plurality of generic dental restorations, means for shaping, means for scanning, and software to be used with means for computing to register digitizing scans obtained with the means for scanning, and for controlling the means for shaping to shape a generic dental restoration. In one exemplary embodiment of this invention: the plurality of generic dental restorations are pre-fired ceramic, the means for shaping is a wet computer numerical controlled (CNC) mill, the means for scanning is a handheld oral scanner, and the software may be used to control a cutting path of the wet computer numerical controlled (CNC) mill in or on a dental restoration.

In one aspect, a method for making a dental restoration on a subject includes the steps of providing at least one dental restoration shell blank, the blank having an exterior surface, a base, and an interior volume open at the base, scanning at least one tooth of interest on the subject to produce a first set of scanning data, and saving the first set of scanning data to an external scan data file, forming the at least one tooth of interest into a shape configured to receive the dental restoration shell blank, scanning the at least one formed tooth of interest to produce a second set of scanning data, and saving the second set of scanning data to an internal scan data file, creating a digital tooth design from the external and internal scan data files, and shaping the exterior surface and the interior volume of the at least one dental restoration shell blank in accordance with the digital tooth design, to produce a finished dental restoration configured to fit over the formed tooth.

The following features may also be incorporated in the above described embodiments.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a schematic diagram which illustrates steps which may be used in methods of the present invention in order to produce dental restorations;

FIG. 2 is a side-view illustrating “pre-scanning” of a patient's mouth;

FIG. 3A is a cut-away side-view illustrating a dental restoration shell blank of the present invention;

FIG. 3B is a bottom (axial) view of the dental restoration shell blank of FIG. 3A; and

FIG. 4 is a side-view which depicts a prepared (“prepped”) tooth of interest, and the scanning thereof.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As contemplated herein, the words “patient” and “subject” are used interchangeably, to refer to an individual undergoing a procedure or method, for example via a system as disclosed herein.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

Some aspects of the present invention may be made using an additive manufacturing (AM) process. Among the most common forms of additive manufacturing are the various techniques that fall under the umbrella of “3D Printing”, including but not limited to stereolithography (SLA), digital light processing (DLP), fused deposition modelling (FDM), selective laser sintering (SLS), selective laser melting (SLM), electronic beam melting (EBM), and laminated object manufacturing (LOM). These methods variously “build” a three-dimensional physical model of a part, one layer at a time, providing significant efficiencies in rapid prototyping and small-batch manufacturing. AM also makes possible the manufacture of parts with features that conventional subtractive manufacturing techniques (for example CNC milling) are unable to create.

Suitable materials for use in AM processes include, but are not limited to, using materials including but not limited to nylon, polyethylene terephthalate (PET), acrylonitrile butadiene styrene (ABS), resin, polylactic acid (PLA), polystyrene, and the like. In some embodiments, an AM process may comprise building a three dimensional physical model from a single material, while in other embodiments, a single AM process may be configured to build the three dimensional physical model from more than one material at the same time.

To overcome the limitations of the prior art, applicant has devised new methods and means for improved production of dental restorations which more easily produce a dental restoration having excellent fit and at a lower cost than has previously been achieved, as exemplified by the following description. Reference will now be made in detail to embodiments encompassed by, and illustrative of, the present invention, including descriptions of illustrative examples which are depicted by the accompanying drawings. Wherever possible, the same reference numbers are used throughout the drawings and refer to the same or similar parts. Furthermore, the present invention is not limited to the specific examples, embodiments or terminology used herein, but rather should be understood to encompass other aspects and equivalents, which will become readily apparent to one of ordinary skill upon review of this disclosure. Advantages, objects, and features of the invention are set forth in part in the description that follows, however additional advantages, objects and features will become apparent to those of ordinary skill in this art, upon consideration of the following, or may be learned from practice of this invention.

The phrase “dental restoration” is used herein to refer to restoring the function of a tooth or teeth by replacing tooth structure (which may be missing, removed, damaged, etc.). A dental restoration includes for example a crown, bridge, implant, inlay, on-lay, etc.

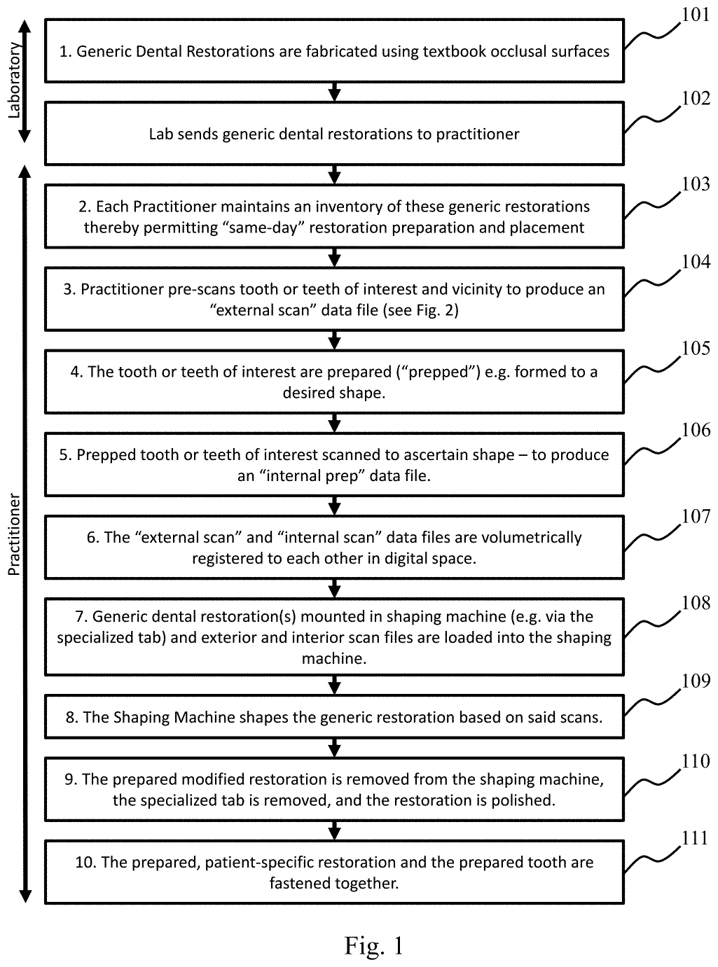

FIG. 1 illustrates process steps of the present invention, which shall be described in greater detail hereinafter, in combination with the other drawing figures and additional descriptions. While the boxes in FIG. 1 are numbered consecutively, it should be understood that additional and intervening steps may also be employed. It should be further understood that in some circumstances, one or more steps enumerated in FIG. 1 may be performed in parallel or in a different order than depicted.

As indicated in FIG. 1, a first step may comprise the fabrication of generic dental restorations (e.g. generic posterior crowns) for example in a facility which manufactures dental restorations (sometime referred to as a “dental laboratory” or “dental lab”) using typical (or “textbook”) characteristics (such as occlusal surfaces) that conform or correspond to standard types that are widely utilized. It is desirable in practicing this invention to utilize generic dental restorations which are a shell (i.e. a blank which defines an interior volume or cavity) as will be described in greater detail hereinafter. More specifically, blanks which define an interior volume configured and dimensioned to fit over a prepared tooth or teeth of interest, as will be described in further detail hereinafter. These generic dental restorations are preferably made of sintered zirconia, but may also be made of other materials, including but not limited to glass, lithium disilicate, ceramics, polymers, porcelains, Lucite, composites known in the art, including ceramics composites, gold, etc.

In some embodiments, a generic dental restoration may be made from any material specified in ISO 6872, or ISO-13485:2015, both incorporated herein by reference. In other embodiments, any material may be used which has properties including a mean flexural strength of at least 50 MPa (for example in a class 1 dental ceramic, defined as a monolithic ceramic for single-unit anterior prostheses, veneers, inlays, or on-lays adhesively cemented, or as a ceramic for coverage of a metal framework or ceramic substructure), at least 100 MPa (for example in a class 2 dental ceramic, defined as a monolithic ceramic for single-unit anterior or posterior prostheses adhesively cemented, or as a partially or fully covered substructure ceramic for single-unit anterior or posterior prostheses adhesively cemented). In some embodiments, a mean flexural strength may be at least 300 MPa, for example in a class 3 dental ceramic, defined as a monolithic ceramic for single-unit anterior or posterior prostheses adhesively or non-adhesively cemented, and for three-unit prostheses not involving molar restoration, or a partially or fully covered substructure for single-unit anterior or posterior prostheses and for three-unit prostheses not involving molar restoration adhesively or non-adhesively cemented.

In some embodiments, a mean flexural strength may be at least 500 MPa, for example in a class 4 dental ceramic, defined as a monolithic ceramic for three-unit prostheses involving molar restoration, or a partially or fully covered substructure for three-unit prostheses involving molar restoration. In some embodiments, a mean flexural strength may be at least 800 MPa, for example in a class 5 dental ceramic, defined as a monolithic ceramic for prostheses involving a partially or fully covered substructure for four or more units or fully covered substructure for prostheses involving four or more units.

In some embodiments, a chemical solubility may be at least 100 μg/cm², for example in a class 1, 2, 3, 4, or 5 dental ceramic, at least 2000 μg/cm², for example in a class 2, 3, or 4 dental ceramic. In some embodiments, a fracture toughness may be at least 0.7 MPa√m, for example in a class 1 dental ceramic, at least 1.0 MPa√m, for example in a class 2 dental ceramic, at least 2.0 MPa√m, for example in a class 3 dental ceramic, at least 3.5 MPa√m, for example in a class 4 dental ceramic, or at least 5.0 MPa√m, for example in a class 5 dental ceramic.

In one exemplary embodiment, the process of the present invention may be practiced with zirconia generic restorations, limiting modifications to shaping/machining of zirconia generic restorations. Economies are realized which make of the steps of this process more economical, and this process facilitates production of a patient-specific zirconia crown, fitted in a clinical setting—during only one visit, which is an advantage over the multiple visit, multi-week methods (with some mal-fitting) of the prior art. Further, generic dental restorations of the present invention may be made with a variety of different characteristics, such as by varying one or more one or more of: shape, size, shade (color), material, etc.

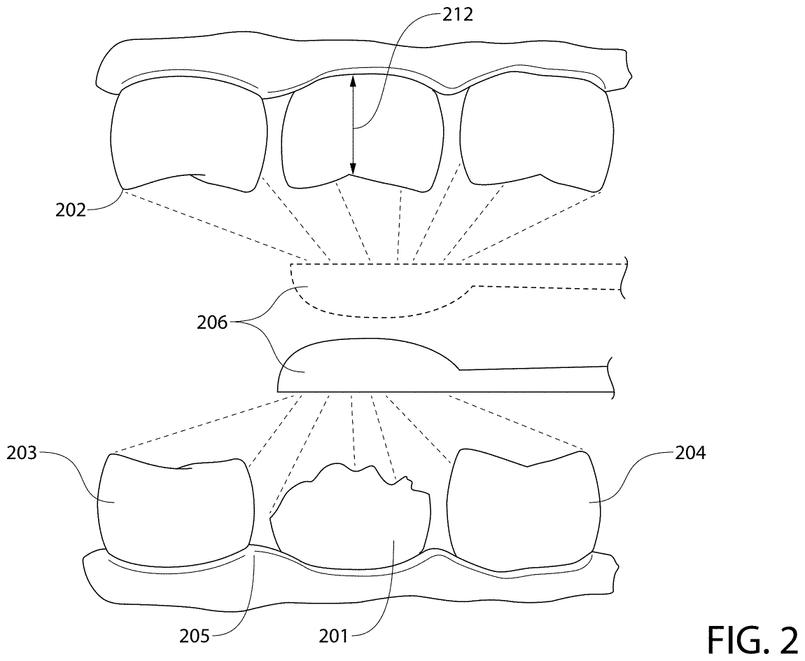

The generic dental restorations may in some embodiments be fabricated with greater height (a.k.a. length) than is anticipated to be needed, so that the final shape of their interior and margins may be fit specially to the individual patient's requirements (i.e. the starting height/length of the generic restoration is long enough for every mouth, and is shaped/machined down to fit the specific patient). For the sake of clarity, the direction in which tooth-height (a.k.a. length) is measured is indicated by line 212 in FIG. 2.

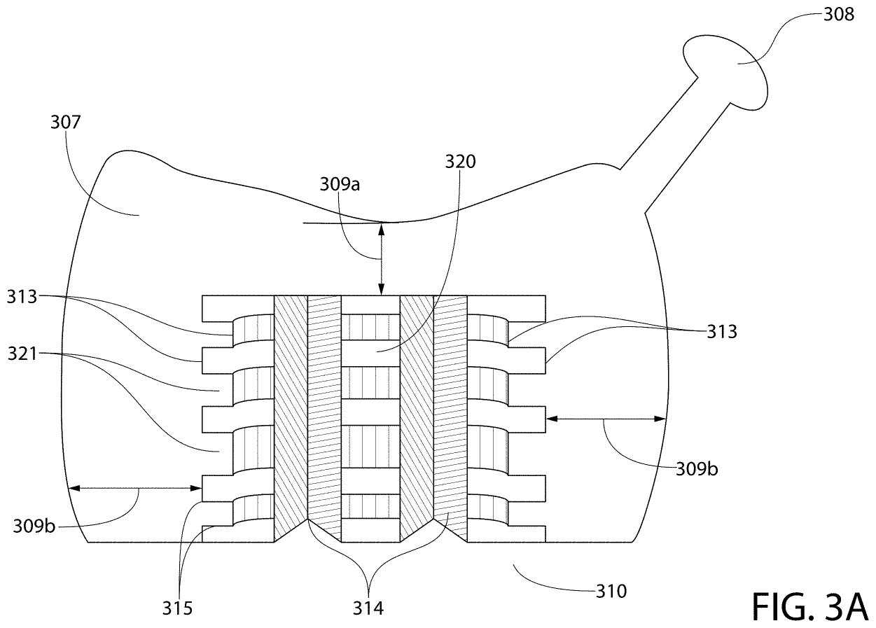

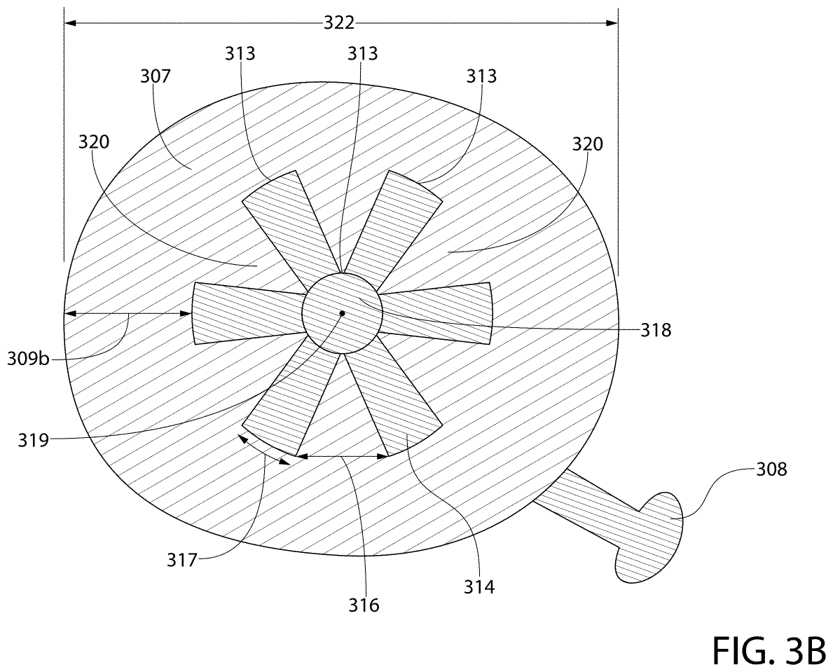

As illustrated in FIG. 3A and FIG. 3B, each generic dental restoration blank shell 307 includes a specialized tab (a.k.a. “dog-bone”), one exemplary embodiment of which is shown as 308 in both FIG. 3A and FIG. 3B. Tab 308 is used to facilitate the manufacturing of the dental restoration and may be used for example as a handle to securely and accurately hold/mount the restoration during manufacturing. Although the tab is depicted has having a dog-bone shape in FIG. 3A and FIG. 3B, it is understood that such tabs may have a different shape or mounting point on a generic dental restoration blank shell. In some embodiments, more than one tab may be used as a grasp to spin the restoration while in the scanner when the scan takes place.

The generic dental restoration blank shell 307 defines an interior volume/cavity 310 which is open at the base of the dental restoration blank shell 307. The interior volume may be dimensioned and configured to fit over a prepared tooth of interest, as will be described in greater detail hereinafter. The generic dental restoration shell 307 may be shaped to define top wall thickness 309a and side-wall thicknesses 309b (shown in both FIG. 3A and FIG. 3B). The top wall thickness 309a may in some embodiments be about 1.5 millimeters and side-wall thicknesses may in some embodiments be about 3 millimeters. Because tooth preparation will vary from patient to patient, a side-wall thickness of about 3 mm allows enough material to accommodate all dental needs. Depending on the material used, a top wall thickness of about 1.5 mm is the minimum recommended thickness for proper strength of the occlusal surface. It is understood that top wall thickness may vary for different materials having different strengths and characteristics. Generally, it is advisable to have more solid material and less cement, to achieve the best bonding of the dental restoration (e.g. better load transfer and longevity).

Also, interior surfaces 313 of the generic dental restoration shell may in some embodiments define the interior volume 310, a first set of essentially parallel grooves 314 defined by a first set of essentially parallel ridges 320 which extend essentially vertically (in the orientation shown), and a second set of essentially parallel horizontal (in the orientation shown) grooves 315 defined by a second set of essentially parallel ridges 321 which extend around the interior surfaces 313. These two sets of grooves and ridges cross/intersect, and thereby form what may be termed internal cross-hatching, or rugae. This cross-hatched/rugae configuration provides the advantages of expediting the entire process, facilitating and reducing the time required for removal of material (e.g. milling) from this interior volume, increasing retention capability because the adhesive will encase around the cross-hatching/rugae, and providing more surface-area to which adhesives may adhere. While two groves 314 of the first set of grooves are illustrated in FIG. 3A, and six grooves 314 of the first set of groves are illustrated in FIG. 3B, it should be understood that the number of grooves may be varied, for example according to the type of materials used, the strength needed, manufacturing expediencies, etc. Analogously, while five grooves 315 of the second set of grooves are illustrated in FIG. 3A, the number of these grooves 315 may be varied as well.

Exemplary dimensions include the following. The width of the base 316 of each of the first set of ridges 320 may be about 2 millimeters. The spaces 317 between the bases of adjacent ridges 320 may be about 1 millimeter. Initially, the innermost portions/points of the first set of ridges 320 may be spaced about 1 millimeter outward from a center point 319 of the dental restoration (i.e. the innermost portions/points of the first set of ridges 320 may lay along a center-circle 318 with a diameter of about 2 millimeters). The width of the cross-section of the dental restoration 322 may, for example, be about 8 millimeters. Such internal feature/ridge spacing is wide enough to allow for some interdigitating with the cement. Also, in some embodiments it is desirable that there be a taper at the bottommost edge of the dental restoration, in order for it to fit on to the margin shelf that the practitioner has prepared on the patient's tooth. This margin shelf is not uniform, and the excess material on the bottom of the dental restoration accommodates this lack of uniformity. Also, excess wall thickness may be provided to make allowances for variables in the tooth preparation by the practitioner.

Referring again to FIG. 1, these generic dental restorations may be sent from the dental lab to a dental practitioner's office/facility in step 102, where an inventory of a variety of these generic restorations is maintained, so that the practitioner may select from the inventory one or more restorations with characteristics which best suit an individual patient and provide “same day” restoration preparation and placement.

As would be understood by one of skill in the art, it is desirable that there by no space between adjacent teeth because such a space allows for food to become trapped between teeth where its continued presence can breed harmful bacteria. For this reason, a variety of sizes and shapes of generic dental restoration are necessary for different patients, because a tooth at the same position is not the same size for all individuals. In some embodiments, generic dental restorations may have a smaller number of variants, for example youth sizes, adult sizes, male sizes, female sizes, or the like. In other embodiments, generic dental restorations may have a range of sizes from small to large, and an initial measurement may be used to determine the best generic dental restoration for a particular patient. In some embodiments, a dentist, dental assistant, or other clinician may use a measuring device, for example a caliper, to obtain an accurate measurement of the space or size of the needed restoration intraorally.

The tooth/teeth of interest, and its vicinity, may be scanned using “means for scanning” known in this art which may conveniently be hand-held (FIG. 1, Step 104), the tooth/teeth is digitally “repaired,” and this data file saved as an “external prep” file. As this scan may be acquired before dental work is performed, this step may be termed a “pre-scanning.” Various steps in this process, including such pre-scanning, may be performed by a variety of dental care professionals, including but not limited to a dentist, dental assistant, or other dental care practitioner. Examples of suitable means for scanning known in the art include devices such as: the iTero® Element Intraoral Scanner (http://www.itero.com/en-us) from Align Technology Inc., San Jose Calif.

As illustrated in FIG. 2, this pre-scanning may include obtaining information regarding not only the tooth or teeth of interest (for example tooth 201), but also scanning of its vicinity, including: opposing tooth/teeth 202, adjacent teeth 203 and 204 and/or adjacent surfaces 205. This scanning per se, may be accomplished by various know methods and devices. However, one such method is illustrated in FIG. 2 wherein a scanning device 206 is inserted into a patient's mouth and then used to scan the tooth/teeth of interest and the surrounding region, and subsequently the scanning device 206 is used (as shown in phantom) to scan the opposing teeth and the surrounding region. This pre-scanning may include obtaining information regarding a variety of characteristics, including but not limited to the required dental restoration diameter, desired final occlusal surface, contacts, intra-periodontal depth, ascertaining desired final restoration height, shape, size, anatomy, shade (color), etc. and defects and/or deficiencies such as those caused by weakness, wear, decay, breakage, misshapenness, discoloration or tooth loss—such as broken or missing cusp(s), flattened or worn occlusion(s), tooth alignment such as teeth out of occlusion, undersized tooth, broken tooth, weak contact, etc.

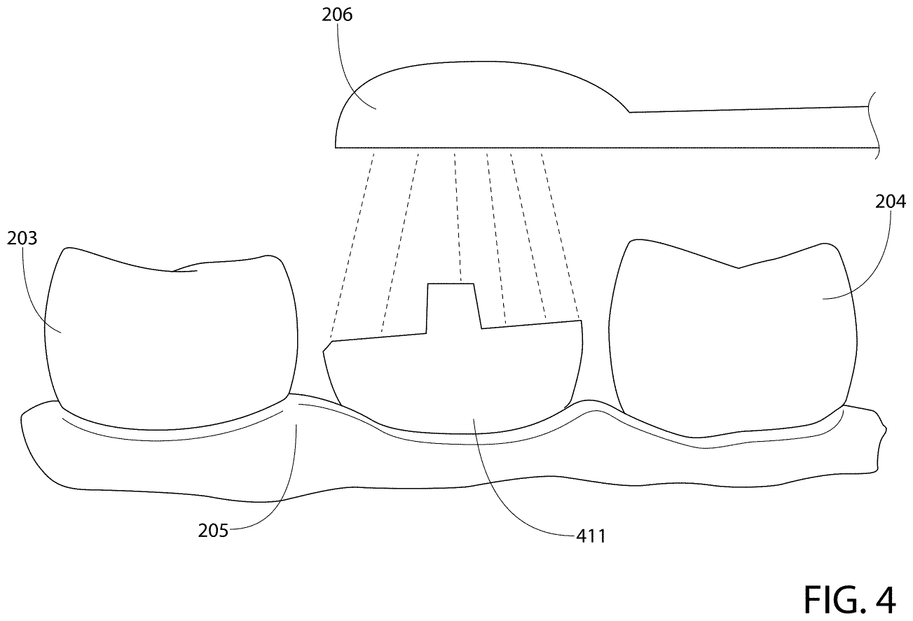

As indicated in FIG. 1, step 105 the tooth or teeth of interest 201 is prepared (i.e. “prepped”) for example by being formed into a desired shape depending on the patient's individual needs, using methods known in this field. This typically produces a “prepped” tooth having a trapezoidal shape, and a shoulder of original tooth left at the level of the gum margins for the dental restoration to sit upon. One exemplary such “prepped” tooth of interest is designated 411 in drawing FIG. 4. In some embodiments, prepping a tooth includes the steps of removing bad enamel and/or dentin from the tooth, for example with a drill or other instrument.

Preparing a tooth for a crown in some embodiments requires two visits to the dentist. The first visit may in some embodiments involve examining and preparing the tooth, while the second visit involves placement of the permanent crown. At the first visit in preparation for a crown, a dentist may image the subject, for example with x-rays, to check the roots of the tooth receiving the crown and surrounding bone. If the tooth has extensive decay or if there is a risk of infection or injury to the tooth's pulp, a dentist may perform a root canal first. Before the process of making a crown begins, a dentist may numb the tooth and the gum tissue around it. Next, the tooth receiving the crown is in some embodiments filed down along the occlusal surface and sides to make room for the crown. The amount removed depends on the type of crown used. In some embodiments, if a large area of the tooth is missing (for example due to decay or damage), a dentist may use filling material to “build up” the tooth to support the crown.

In some embodiments, prepping a tooth includes the step of seating a gingival retraction cord (sometimes referred to herein simply as a “cord”) into the gums or between the tooth and the gums. As understood by those skilled in the art, a gingival retraction cord may in some embodiments be used to push the gum tissue away from the prepared margins of the tooth, in order to create or acquire a scan of an accurate impression of the tooth. A gingival retraction cord may in some embodiments resemble yarn on a stick or an advanced type of flossing stick. To create the piece that fits around the tooth to be worked on, pieces of cotton or polyester may be braided together to create a specific diameter. The braided diameter then goes around the tooth. A gingival retraction cord may be used to push the gum tissue away from the prepared margins of the tooth, in order to create an accurate impression of the teeth. In some situations, the gum can impede the collection of an accurate impression of a tooth, and so therefore it may in some embodiments be desirable to push the gum out of the way. The use of a gingival retraction cord therefore allows the dentist to focus on preparing the tooth.

As also illustrated in FIG. 4, in FIG. 1 step 106, the prepped tooth of interest 411 is then scanned using scanning device 206 in order to capture information regarding its shape, and this data file is saved as an “internal prep” file. This information may then be used in shaping the dental restoration for joining with the prepped tooth of interest, and therefore only the prepped tooth of interest 411 needs to be scanned in this step (i.e. not opposing or adjacent teeth).

As referenced in FIG. 1 Step 107, the practitioner's facility employs computing means which are known in the art, which runs/executes software, which volumetrically registers the “external scan” and the “internal scan” to each other in digital space. This co-registered “digital tooth” is also registered to the specialized tab or tabs (e.g. dog-bone 308). This may be accomplished using CAD/CAM software (i.e. Computer-Aided Design or Computer Aided Manufacturing) which functions to auto-design a dental restoration which addresses the issues and needs of the individual patient (e.g. takes input data, uses best fitting algorithms to calculate the differences between starting and desired finished shapes, and then outputs the controlling commands to the means for shaping).

When a portion of the tooth or teeth of interest is missing, this software may create the optimum dental restoration shape required using a method which may in some embodiments include the step of assessing what is missing from the tooth, and hence computing what the shape of the intact tooth had been. More specifically, the software identifies and analyzes the patient's dental issues and needs, and based thereon, creates a design employing the aforementioned computing device, and associated devices such as a video-display, input means (such as a keyboard, mouse, touch screen, and/or voice commands), etc. This final design will correct issues and/or address the dental needs of the individual patient, including those pertaining to any of the aforementioned characteristics, and thereby provide proper: occlusion, contact, shape, size, anatomy, color (shade), etc.

In FIG. 1 step 108, the generic dental restoration is mounted into suitable means for shaping. For example, the restoration may be held or mounted in a shaping machine via the specialized tab or tabs 308. Suitable means for shaping include, but are not limited to, mills, such as wet mills which are computer numerical controlled (CNC), installed in the practitioner's facility. This “digital tooth” design is then used by the computing means to control means for shaping, which shapes both the exterior and interior volume 310 to fit onto the prepared tooth 411, with proper dimensions, height and margin outline, in FIG. 1 step 109. It is desirable that this shaping step includes shaping each blank to define an “extended height” which is greater than said aforementioned desired final height. It is desirable that this extended height be about one millimeter more than the desired final height. More specifically, the generic dental restoration is oversized in height, and the desired final height would potentially differ from a number 7 versus a number 8 tooth, male to female, etc. The desired final height is determined in the scanning step, and then shaped/machined in the final shaping step.

Although subtractive manufacturing processes are discussed for use in the shaping step to remove material from the generic dental restoration, it is understood that in some embodiments of the invention, additive manufacturing methods may be used alternatively or additionally to add material to the generic dental restoration, either additionally or alternatively. For example, in some embodiments, a polymer crown may be 3D printed, for example from any polymer suitable for use in a dental crown, or for example any polymer approved by the FDA for intraoral crown seating. In some embodiments, an additive manufactured crown may be made from a ceramic or other material using various other AM processes, including for example via SLA.

In some embodiments, some or all of a custom forming process may be performed while a subject is waiting in the office, for example to reduce total overall time that a subject spends in the dental chair (“chair time”). For example, in some embodiments, a polymethyl methacrylate (PMMA) generic crown may be milled or partially milled while measurements are being taken of the subject's teeth. For example, in one embodiment, an outer surface of the PMMA generic may be milled, for example with a CNC mill, while the dimensions for the inner surface are measured. The inner surface may then later be milled to the correct shape after the measurements are made. With such processes, it is possible in some embodiments to fit a subject with a permanent crown in a single visit. Fitting a crown in a single visit instead of the typical two is advantageous because the extra chair-time from the unused second visit could instead be used to see new patients, which creates more business for the dental office. A patient might prefer to have a single visit for efficiency, and to avoid time spent with a less durable temporary crown, which take 1 to 2 more visits to replace with a new permanent crown.

Although the above example describes a PMMA generic crown, it is understood that a custom formed crown of the present disclosure may comprise any material disclosed herein or known in the art. In some embodiments, a custom formed crown may comprise a plurality of materials.

As indicated in FIG. 1 step 110, after the aforementioned shaping step, the prepared modified restoration is removed from the means for shaping, then, where a specialized tab 308 is included, the specialized tab 308 is removed (for example by cutting or milling this tab off), and the dental restoration is polished. More specifically, polishing occurs after final shaping and removal of any specialized tab 308. The dental restoration is polished so that the finished restoration will be smooth enough that the machining areas are not detectable by the patient's tongue.

As illustrated in FIG. 1 step 111, the prepared, patient specific, dental restoration and the prepared tooth of interest are then fastened (e.g. adhered or cemented) together, using means for fastening.

Some embodiments of a method of the present invention comprises steps which may be performed by a dental practitioner's office/facility as discussed above in step 102 of FIG. 1. One exemplary method of the present invention comprises the steps of providing at least one dental restoration shell blank as discussed elsewhere in the present disclosure, the blank having an exterior surface, a base, and an interior volume open at the base. In some embodiments, the dental practitioner's office maintains a supply of such dental restoration shell blanks of varying size and shape. A tooth of a subject and/or the vicinity of one or more teeth of the subject may then be scanned as discussed above in step 104 of FIG. 1, to produce an external scan of the tooth and, in some embodiments, its vicinity. The tooth of interest may then be formed into a shape configured to receive the eventual dental restoration, for example as discussed in step 105 of FIG. 1. The formed tooth may then be scanned again, in order to obtain a precise model (or “internal scan data”) of the shape that will mate with the internal volume of the dental restoration, as discussed in step 106 of FIG. 1. A digital tooth design may then be created from the external scan of the tooth and the internal scan data, providing a 3D CAD model of a finished dental restoration, including an external surface, for example an external surface closely approximating the shape of a healthy tooth in the position of the tooth of interest, and an internal volume within the external surface, and open at the base of the model, shaped to mate with the formed tooth as shaped in step 105. The digital tooth model may then be used to form the dental restoration shell blank, for example via one or more subtractive and/or additive manufacturing techniques. The formed dental restoration shell blank will then be a finished dental restoration configured to fit over the formed tooth.

Having described the invention in detail, those skilled in the art will appreciate that, given the disclosure herein, modification may be made to the invention without departing from the spirit and scope of the inventive concept. It is not intended that the scope of this invention be limited to the specific and preferred embodiments illustrated and described.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### INDEX OF ELEMENTS IDENTIFIED BY A NUMERAL

101-111—Method steps
201—Tooth of Interest
202—Opposing tooth or teeth
203 and 204—Adjacent teeth
205—Adjacent surfaces
206—Scanning device
307—Generic dental restoration blank shell
308—Specialized tab (a.k.a. “dog-bone”)
309a—Dental restoration top-wall thickness
309b—Dental restoration side-wall thicknesses
310—Dental restoration interior volume/cavity
312—Direction in which tooth height (a.k.a length) is measured
313—Interior surfaces which define the interior volume
314—First set of grooves
315—Second set of grooves
316—The width of the base of each of the first set of ridges 320
317—Spaces between the bases of adjacent ridges 320
318—Center circle
319—Center point of the center circle, and center of the dental restoration
320—A first set of essentially parallel ridges
321—A second set of essentially parallel ridges
411—Prepared (“prepped”) tooth

What is claimed is:

1. A method for making one or more dental restorations, including the steps of:
fabricating at least one generic dental restoration shell blank, each said blank defining an exterior, an interior volume within said exterior, and a base, said interior volume being open at said base, and said interior volume being dimensioned and configured to fit over a prepared tooth of interest;
providing said at least one generic dental restoration to a dental practitioner's facility;
scanning at least one tooth of interest and the vicinity thereof, and saving this scanning data to an external scan data file;
preparing said at least one tooth of interest by forming each said tooth into a desired shape depending on the patient's individual needs;
scanning each prepared at least one tooth of interest and saving this data to an internal scan data file;
volumetrically registering said external scan and said internal scan to each other in digital space to create a digital tooth design;
shaping both said exterior and said interior volume of each said at least one generic dental restoration in accordance with said digital tooth design, so that said interior volume of each will fit over a said prepared tooth; and
fastening at least one shaped dental restoration to at least one prepared tooth of interest.

2. The method of claim 1, wherein said dental restoration shell blank is fabricated in a facility which manufactures dental restorations.

3. The method of claim 1, wherein said dental restoration blank is fabricated using typical characteristics of teeth.

4. The method of claim 1, wherein said generic dental restoration shell blanks are generic posterior crowns.

5. The method of claim 1, wherein said generic dental restorations consist of at least one material selected from the group consisting of sintered zirconia, composites and gold.

6. The method of claim 1, wherein said step of scanning at least one tooth of interest and vicinity includes gathering information regarding a characteristic selected from the group consisting of required dental restoration diameter, desired final occlusal surface, contacts, intra-periodontal depth, desired final restoration height, shape, size, anatomy, shade, defects caused by weakness, wear, decay, breakage, misshapenness, discoloration, tooth loss, broken cusp, missing cusp, flattened occlusion, worn occlusion, tooth alignment, undersized teeth, broken tooth, and weak contact.

7. The method of claim 1, wherein said interior volume of each said dental restoration is defined by interior surfaces which define a first set of essentially parallel ridges, and a second set of essentially parallel ridges, and wherein said sets of ridges cross in order to define a cross-hatched or rugae interior surface.

8. The method of claim 1, wherein said step of fabricating at least one generic dental restoration shell blank comprises:
ascertaining a desired final height for each said dental restoration; and
fabricating each dental restoration to a height which is greater than said desired final height.

9. The method of claim 1, wherein each said generic dental restoration shell blank defines a specialized tab extending from said exterior surface thereof, and further including the step of utilizing said specialized tab as a handle to hold the dental restoration during manufacturing.

10. The method of claim 9, further comprising the step of removing said tab prior to said step of attaching each said final dental restoration to a said prepared surface.

11. The method of claim 9, wherein the said step of volumetrically registering also includes a step of co-registering said scans with said specialized tab.

12. The method of claim 1, wherein said dental practitioner's facility contains an inventory of a variety of said generic dental restoration shell blanks; and
further comprising the step of a dental practitioner selecting from said inventory a dental restoration shell blank which has characteristics which best suit an individual patient.

13. The method of claim 1, wherein said step of preparing at least one tooth of interest includes removing an element from the group consisting of remnants of an original occlusal surface, caries, damaged areas, and previously applied restorative material.

14. The method of claim 13, wherein said step of removing leaves a tooth having a trapezoidal shape, and a shoulder of original tooth left at a level of gum margins for said dental restoration to sit upon.

15. The method of claim 1, wherein said step of scanning each prepared tooth of interest does not include scanning teeth other than said prepared tooth of interest.

16. The method of claim 1, further comprising the step of using means for computing to design said digital tooth design to be a dental restoration which addresses issues and needs of an individual patient.

17. The method of claim 1, wherein said digital tooth design corrects dental issues or needs of an individual patient, including one of more of: occlusion, contact, shape, size, anatomy, or color.

18. A kit comprising:
a plurality of generic dental restorations;
means for shaping;
means for scanning; and
software to be used with means for computing to register digitizing scans obtained with said means for scanning and for controlling said means for shaping to shape a said generic dental restoration.

19. The kit of claim 18 wherein:
said plurality of generic dental restorations comprise pre-fired ceramic;
said means for shaping comprise a wet computer numerical controlled (CNC) mill;
said means for scanning comprise a handheld oral scanner; and
said software may be configured to control a cutting path of said wet computer numerical controlled (CNC) mill in or on a said dental restoration.

20. A method for making a dental restoration on a subject, including the steps of:
providing at least one dental restoration shell blank, the blank having an exterior surface, a base, and an interior volume open at the base;
scanning at least one tooth of interest on the subject to produce a first set of scanning data, and saving the first set of scanning data to an external scan data file;
forming the at least one tooth of interest into a shape configured to receive the dental restoration shell blank;
scanning the at least one formed tooth of interest to produce a second set of scanning data, and saving the second set of scanning data to an internal scan data file;
creating a digital tooth design from the external and internal scan data files; and
shaping the exterior surface and the interior volume of the at least one dental restoration shell blank in accordance with the digital tooth design, to produce a finished dental restoration configured to fit over the formed tooth.
